(19) United States
(12) Patent Application Publication
(10) Pub. No.: US 2026/0007453 A1
(43) Pub. Date: Jan. 8, 2026

(54) MEDICAL DEVICE WITH DISTAL ROTATION

(71) Applicants: Boston Scientific Scimed, Inc., Maple Grove, MN (US); Boston Scientific Medical Device Limited, Galway (IE)

(72) Inventors: Yin XIA, Shanghai (CN); Kensuke HAYASHI, Penang (MY)

(73) Assignees: Boston Scientific Scimed, Inc., Maple Grove, MN (US); Boston Scientific Medical Device Limited, Galway (IE)

(21) Appl. No.: 19/257,988

(22) Filed: Jul. 2, 2025

(30) Foreign Application Priority Data
Jul. 5, 2024 (CN) 202410906118.4

(51) Int. Cl. A61B 18/14; A61B 17/29; A61B 90/00

(52) U.S. Cl. CPC A61B 18/1447; A61B 2017/2905; A61B 2017/2919; A61B 2090/035

(57) ABSTRACT

A medical device comprising a shaft, an end effector assembly at a distal end of the shaft, and a connection between the end effector assembly and the shaft permitting axial rotation of the end effector assembly relative to the shaft. The connection includes a shaft connector fixed to a proximal end of the end effector assembly and protruding radially inward from an inner surface of the end effector assembly. The connection also includes a stop fixed to a radially outward facing surface of the shaft, distal of the shaft connector. The stop is axially translatable relative to the shaft connector to engage a distally-facing surface of the shaft connector.

### CROSS-REFERENCE TO RELATED APPLICATIONS

This application claims the benefit of priority to Chinese Application No. 202410906118.4, filed on Jul. 5, 2024, which is incorporated by reference herein in its entirety.

### TECHNICAL FIELD

Various embodiments of this disclosure relate generally to medical devices having a rotatable end effector.

### BACKGROUND

In some medical procedures, a physician may require precise manipulation of an end effector. For example, during endoscopic surgeries, a physician may use accessory devices, such as forceps, baskets, nets, etc., to access and treat a site within a patient. The physician or a technician may introduce an accessory device into a working channel of a scope, such as an endoscope, to reach a treatment site within the patient. Once the site is reached, the physician may proceed to provide treatment to the desired site. Currently, devices often do not have the capability to rotate the end effector independently from the shaft or the handle in a precise manner. Imprecise manipulation of the end effector is disadvantageous during such procedures, as it can lead to increased procedure times and difficulty accessing a treatment site or delivering treatment.

This disclosure is directed to overcoming one or more of these above-referenced challenges or other challenges in the art.

### SUMMARY

Aspects of the disclosure relate to, among other things, devices, and methods to enable actuation and rotation of a distal end effector to enable treatment at a target tissue site within a subject (e.g., patient). Each of the aspects disclosed herein may include one or more of the features described in connection with any of the other disclosed aspects.

According to certain aspects of the disclosure, a medical device may comprise a shaft, an end effector assembly at a distal end of the shaft, and a connection between the end effector assembly and the shaft that may permit axial rotation of the end effector assembly relative to the shaft. The connection may include a shaft connector fixed to a proximal end of the end effector assembly and protruding radially inward from an inner surface of the end effector assembly. The connection may also include a stop fixed to a radially outward facing surface of the shaft, distal of the shaft connector. The stop may be axially translatable relative to the shaft connector to engage a distally-facing surface of the shaft connector.

Any of the medical devices disclosed herein may have any of the following features, alone or in any combination. The end effector assembly may include a clevis. A proximal end of the clevis may include a shoulder, and the shaft connector may be coupled to the shoulder of the clevis. A first gap may be defined between the stop and the clevis, and a second gap may be defined between the stop and the shaft connector. The shaft connector may include a ledge, and the ledge may extend radially inward and distally toward the second gap. The shaft connector may translate axially relative to the shaft within the second gap. The stop may rotate relative to the shaft connector. The stop may abut a distal-facing surface of the ledge when the stop translates axially in a proximal direction. The end effector assembly may be rotated when moved by an actuator, and the shaft connector and the clevis may rotate relative to the shaft during rotation. The shaft may comprise a coil and a covering, and the covering may be fixed to an outer surface of a proximal portion of the coil, and a proximal surface of the shaft connector may abut the covering. A distal portion of the coil may have an outer diameter that is less than an outer diameter of the proximal portion of the coil. The stop may have an inner diameter matching the outer diameter of the distal portion of the shaft, and the the stop may be fixed to the distal portion of the coil. The shaft connector may be radially outward of the distal portion of the coil.

Furthermore, the medical device may include a handle piece and an actuator coupled to the handle piece. A wire may be fixedly coupled to the actuator and extend to the end effector assembly for actuation of the end effector assembly. The handle piece may include a distal extension that has a radial protrusion and an annular gap. Additionally, the medical device may include a holder defining a circumferential opening that receives the radial protrusion. An annular ring may be positioned on the shaft and sit within the annular gap in the extension. The handle piece, the actuator, and the wire may rotate relative to the holder and the shaft.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It may be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated herein and constitute a part of this specification, illustrate exemplary aspects of the disclosure and, together with the description, explain the principles of the disclosure.

FIG. 1 is a side view of a medical device, according to aspects of this disclosure;

FIG. 2A is an enlarged view of portions of the handle of FIG. 2B, according to aspects of this disclosure;

FIG. 2B is a cross-sectional view of an exemplary handle of the medical device of FIG. 1, according to aspects of this disclosure; and

FIGS. 3A and 3B are a side cross-sectional view and a perspective view respectively of an exemplary end effector assembly, according to aspects of this disclosure.

FIG. 4A is a side view of a medical device, according to aspects of this disclosure.

FIG. 4B is a side cross-sectional view of an exemplary portion of a handle assembly of the medical device of FIG. 4A, according to aspects of this disclosure.

### DETAILED DESCRIPTION

Aspects of the disclosure include devices and methods to enable actuation and rotation of a distal end effector of a medical device to enable treatment at a target tissue site within a subject (e.g., patient). The ability to manipulate (including rotate) the end effector in a precise manner enables a user to grasp or otherwise manipulate a target more efficiently, thereby reducing procedure time, more precise grasping/manipulation of tissue, and a decreased volume of blood loss, all of which may also result in better patient outcomes.

The medical device may be introduced into the body without a delivery device or via a delivery device. The delivery device may be a catheter, a scope (endoscope, bronchoscope, colonoscope, duodenoscope, etc.), a tube, a sheath, or other like device, inserted into a body cavity or lumen, for example the GI tract, via a natural orifice. The orifice can be, for example, the nose, mouth, or anus, and the placement can be in any portion of the GI tract, including the esophagus, stomach, duodenum, large intestine, or small intestine. Delivery and placement also can be in other body lumens or organs reachable via the GI tract, any natural opening, any other body tract, or any bodily incision.

Reference will now be made in detail to aspects of the disclosure, examples of which are illustrated in the accompanying drawings. Wherever possible, the same or similar reference numbers will be used through the drawings to refer to the same or like parts. The term “distal” refers to a portion farthest away from a user when introducing a device into a patient. By contrast, the term “proximal” refers to a portion closest to the user when placing the device into the subject. As used herein, the terms “comprises,” “comprising,” or any other variation thereof, are intended to cover a non-exclusive inclusion, such that a process, method, article, or apparatus that comprises a list of elements does not necessarily include only those elements but may include other elements not expressly listed or inherent to such process, method, article, or apparatus. The term “exemplary” is used in the sense of “example,” rather than “ideal.” As used herein, the terms “about,” “substantially,” and “approximately,” indicate a range of values within +/−10% of a stated value.

Examples of the disclosure may relate to devices and methods for performing various medical procedures and/or treating portions of the large intestine (colon), small intestine, cecum, esophagus, any other portion of the gastrointestinal tract, and/or any other suitable patient anatomy (collectively referred to herein as a “target treatment site”). Various examples described herein include single-use or disposable medical devices. Any structures of the medical devices described herein can be made of biocompatible materials, including biocompatible polymers, rubbers, plastics, and the like.

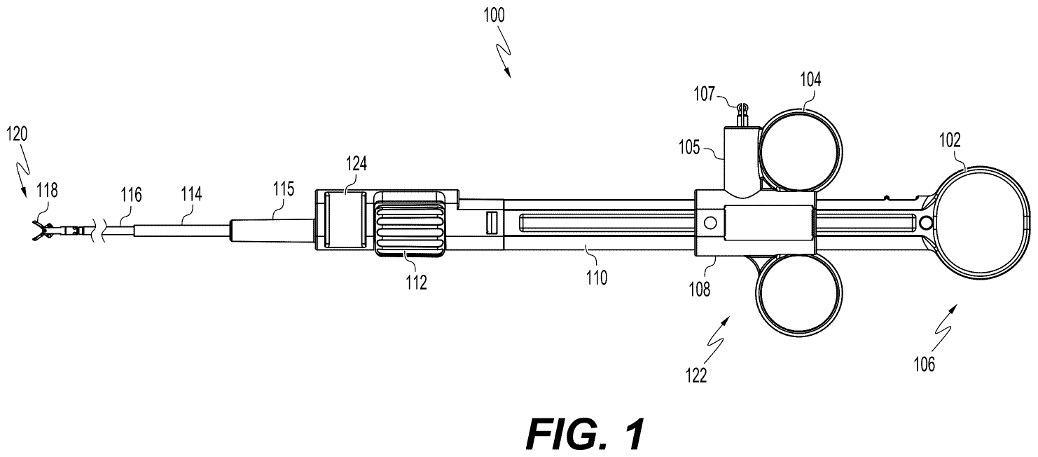

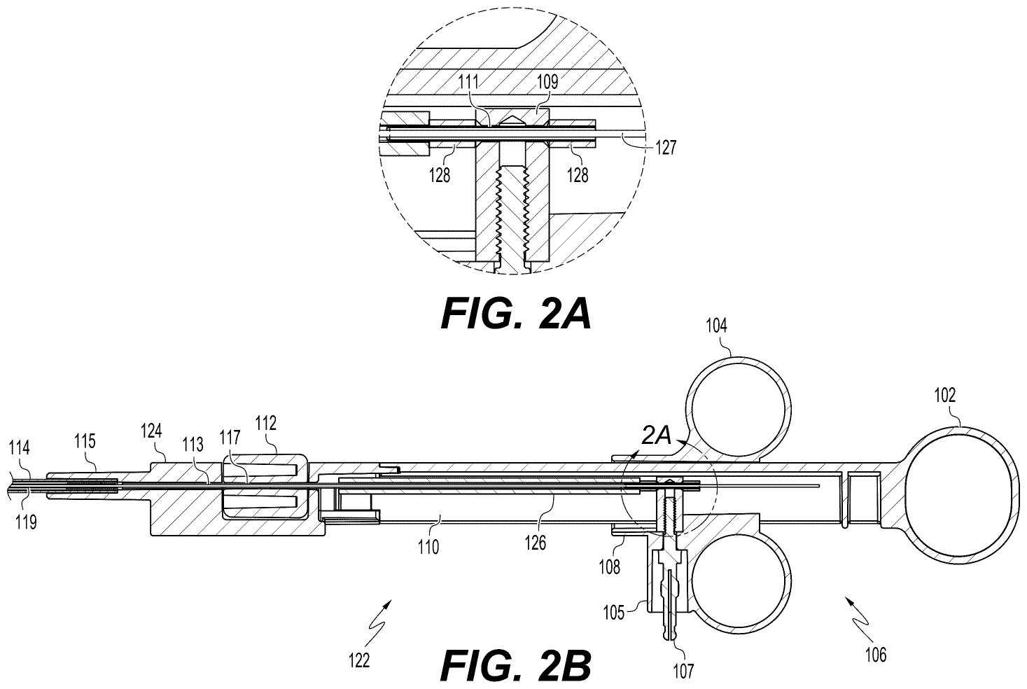

Embodiments of this disclosure relate to a rotation structure proximate to the distal end of a medical device, the rotation structure permitting rotation of an end effector of the medical device (e.g., 360 degrees) about its axis and relative to the shaft of the medical device. FIGS. 1, 2A, and 2B depict a medical device and its handle that may be used in combination with that rotation structure. Those Figures are from PCT International Publication Number WO 2023/017313. The following description of those Figures is also present in that PCT publication, the entire contents of which are incorporated by reference herein.

FIG. 1 is a general depiction of a medical device 100 in accordance with examples of this disclosure. Medical device 100 includes a proximal end 106 and a distal end 120. A handle assembly 122 is located at or proximal to proximal end 106 and includes one or more actuators 108, 112. FIG. 2B shows a cross-sectional view of handle assembly 122, flipped over relative to its position in FIG. 1. Actuator 108 may be located at or near a proximal end of handle assembly 122. Actuator 108 controls the actuation (e.g. opening and closing) of end effectors of medical device 100 and provides electrical current to the end effectors, as will be described. Actuator 108 may include one or more finger slot(s) 104 extending outwardly from actuator 108. The one or more finger slot(s) 104 may be located at or near a proximal end of actuator 108. Alternatively, the one or more finger slot(s) 104 may be located at or near a distal end of actuator 108. The one or more finger slot(s) 104 may be any suitable shape and size to enable a user to insert a finger through the slot 104. For example, the one or more finger slot(s) 104 may be circular (as shown), triangular, rectangular, cylindrical or any shape commonly known in the art. Actuator 108 may also include an electrical insert 107 within a cylindrical protrusion 105, both of which extend outwardly from a central portion of actuator 108. The electrical insert 107 and cylindrical protrusion 105 may be located at or near a distal end of actuator 108. Alternatively, the electrical insert 107 and cylindrical protrusion 105 may be located at or near a proximal end of actuator 108.

Actuator 112 may be located at or near a distal end of handle assembly 122. Actuator 112 may include a knob, trigger, button, switch, pneumatic control, or any other actuator known in the art and may control the rotation of a distal end of medical device 100. Actuator 112, its interaction with other components of device 100, and its use are described further herein. Handle assembly 122 may further include a handle piece 110. Handle piece 110 may include a track on which actuator 108 is moveably coupled to such that actuator 108 may translate in a first direction, towards a distal end of handle assembly 122, and in a second direction, towards a proximal end of handle assembly 122. For example, handle piece 110 may include a central longitudinal slot through which a portion of actuator 108 extends. Actuator 108 translates longitudinally within that slot in the first and second directions. The movement of actuator 108 in a first direction and in a second direction results in the actuation or de-actuation of a distal end effector assembly 118 by means of, for example, a connection of an elongate member, such as a cable 126 (shown in FIG. 2B), between the actuator 108 and the distal components. Cable 126 may include an electrically conductive wire covered by an insulative sheath 119. The connections of cable 126 to various components are described further herein.

Handle piece 110 includes a finger slot 102 located at or near a proximal end of handle piece 110. Finger slot 102 may be shaped similarly or differently as compared to the one or more finger slot(s) 104. For example, the one or more finger slot(s) 104 may be circular, and the finger slot 102 may be circular, ovular, or rectangular.

Handle assembly 122 may also include a handle piece 124. Handle piece 124 may be fixedly coupled to a distal end of handle piece 110 or may be integrally formed with handle piece 110. Handle piece 124 may have a recess that accepts the actuator 112 and related mechanisms to facilitate the rotation of a distal end of medical device 100, to be described further herein. A cylindrical protrusion 115 extends from a distal end of handle piece 124. The protrusion 115 may provide structural support to a strain relief 114 and/or a shaft 116. As shown in FIG. 2B, strain relief 114 may extend into and terminate within protrusion 115 by means of glue, overmold, press-fit, or any other means commonly used in the art. Additionally, shaft 116 may extend through strain relief 114 and terminate within protrusion 115 or handle piece 124. Similar to strain relief 114, the termination of shaft 116 within strain relief 114 or protrusion 115 may be accomplished by means of glue, overmold, press-fit, or any other means commonly used in the art.

Still referring to FIG. 1, a shaft 116 of device 100 extends from a distal end of handle assembly 122 to the distal end 120 of device 100. Distal end 120 includes a distal end effector assembly 118, to be described further therein. Shaft 116 may be a tube having sufficient length to access sites within the body. Additionally, shaft 116 may have sufficient flexibility to traverse tortuous anatomy. Shaft 116 can be made of flexible materials, rigid materials, or any combination thereof.

FIGS. 2A and 2B show a cross-section of handle assembly 122, showing components that are interior of handle pieces 110 and 124. Electrical insert 107 extends through protrusion 105 and into actuator 108. Electrical insert 107 is coupled, for example via a screw connection, to an electrically conductive electrical connector 109. Electrical connector 109 includes a thru-hole 111 that extends in a direction of the longitudinal axis of handle assembly 122. The proximal portion of the electrically conductive wire 127 of cable 126 (exposed from its insulative sheath 119 at its proximal portion) may extend through thru-hole 111. Between the electrical connector 109 and the wire 127, there is a small gap to enable axial rotation of cable 126 (and its wire 127) relative to electrical connector 109. During this rotation, electrical contact is maintained between the cable 126, its wire 127, and the electrical connector 109. At least one or more cannula(s) 128 may be fixedly coupled to the outer diameter of wire 127. The one or more cannula(s) 128 may abut a distal end and/or a proximal end of the electrical connector 109, thereby preventing axial movement of wire 127 (and its cable 126) relative to the electrical connector 109. The electrical connector 109 provides an electrical interface between electrical insert 107 and the one or more cannulas 128 and wire 127 such that, when the electrical insert 107 is energized or activated (receives electrical current from a connected current source), the one or more cannula(s) 128 and wire 127 are also energized or activated. The connection between these components may provide the electrical current to the distal end effector assembly 118 of medical device 100.

Actuator 112 may be nested in and moveably coupled to handle piece 124 in a manner permitting axial rotation of actuator 112 relative to handle piece 124 but limiting axial movement of actuator 112 relative to handle piece 124. A thru-hole 117 extends axially from a proximal end of actuator 112 to a distal end of actuator 112 through which cable 126 extends. The thru-hole 117 may be rectangular and receive a same or similarly shaped tube 113 coupled to the outside of cable 126. Tube 113 may be coupled to cable 126 by means of a crimp, glue, or other means commonly known in the art. The configuration/connections between cable 126, thru-hole 117, tube 113, and actuator 112 enables rotation of end effector assembly 118 (shown in FIG. 1), when actuator 112 is rotated. For example, when actuator 112 is rotated clockwise, tube 113 and cable 126 rotate accordingly, thereby subsequently rotating an end effector assembly 118 coupled to a distal end of cable 126. Similarly, when actuator 112 rotates counterclockwise, tube 113 and cable 126 rotate accordingly, thereby subsequently rotating an end effector assembly 118 coupled to a distal end of cable 126. As mentioned above, cable 126 may have an insulative sheath 119, extending from a distal end of cable 126 to a proximal end. Insulative sheath 119 may terminate within tube 113 or a location distal to actuator 112 (e.g. within shaft 116), to expose wire 127 and permit the connections of wire 127 to other components within handle assembly 122.

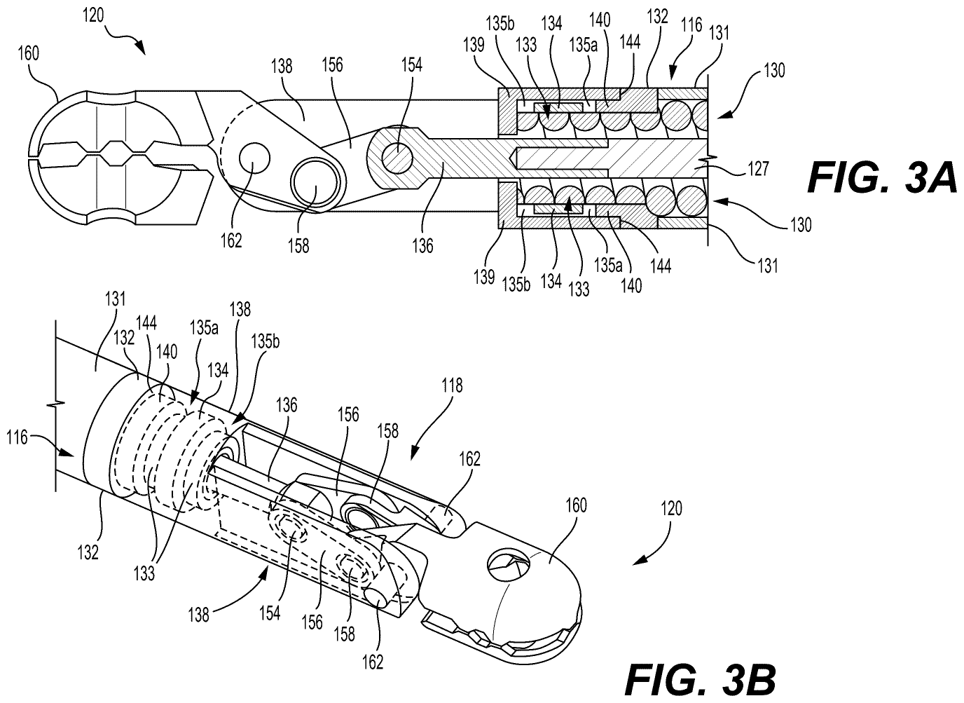

FIGS. 3A-3B show components of an exemplary distal end effector assembly 118 and distal components of shaft 116 that form a rotational structure permitting the rotation of end effector assembly 118 360 degrees about its axis and relative to shaft 116 of the medical device.

FIG. 3A is a cross-sectional view of distal end effector assembly 118. Distal end effector assembly 118 may include a variety of components, including tools and parts to connect the tools to other parts of medical device 100 and permit the various functions of those tools. Exemplary tools include but are not limited to, a tissue grasper (such as an electrocautery/coagulation grasper as shown in the Figures), a knife, biopsy forceps, scissors, a retrieval device (such as a net or a basket), an electrocautery tool, etc. The electrocautery/coagulation grasper, shown in the Figures, enables a user to grasp tissue (particularly bleeding tissue, for example) and deliver energy for procedures where coagulation is desirable to reduce bleeding.

During such procedures and when otherwise necessary, device 100 may be coupled to an electrical source to provide monopolar or bipolar energy to distal end effector assembly 118. As mentioned above, handle actuators (e.g., actuators 108, 112) and related mechanisms can control the rotation and actuation (e.g., open/close movement) of distal end effector assembly 118. For example, a connection between the actuators and the distal components, such as provided by cable 126, transmits the action of actuators 108, 112 to the respective functionality at the distal end of distal end effector assembly 118, the mechanisms of which are described in further detail below.

Aspects of the distal end of shaft 116 will now be discussed in further detail. Shaft 116 comprises a coil 130, having a plurality of winds of circular cross-sectioned wire, and a covering 131 fixed to the outer surface of coil 130. Coil 130 includes a lumen extending therethrough for containing cable 126. Wire 127 of cable 126 extends distally past a distal-most end of covering 131 of shaft 116. A distal portion of coil 130 may have an outer diameter that is less than the outer diameter of the coils 130 of a proximal portion of coil 130 (i.e., a portion of coils 130 covered by covering 131). The distal portion of coil 130 may include a series of shaved coils 133. The distal portion of coil 130 may be a portion of coil 130 (i.e., shaved coils 133) interfacing with an annular stop ring 134 or an annular shaft connector 132, as described below. Shaved coils 133 may be machined through any suitable process, reducing material from the radially outer surface of shaved coils 133, so that the outer surfaces of shaved coils are flat or approximately flat.

Shaved coils 133 are machined (or otherwise manufactured) such that shaved coils 133 interface with annular ring 134 that acts as a stop, as will be described further herein. Ring 134 may have an inner diameter matching or approximately matching an outer diameter of shaved coils 133. In embodiments, ring 134 may be fixed to a radially outward facing surface of shaft 116. For example, ring 134 may be welded, glued, or otherwise fixed to the distal portion of shaft 116 and/or shaved coils 133. Furthermore, ring 134 may be positioned such that a variably-sized gap 135a is defined between ring 134 and a ledge 140, and a variably-sized gap 135b is defined between ring 134 and an inner surface of a clevis 138, described below. Ring 134 may translate within gap 135a and prevent the detachment of shaft 116 from end effector assembly 118, as will be described.

A distal end of shaft 116 is movably coupled to distal end effector assembly 118. A distalmost end of coil 130 of shaft 116 may abut an inner surface of a proximal end 139 of clevis 138, such that coil 130 sits within clevis 138, as shown in FIG. 3A. In other words, a distalmost face of coil 130 (i.e., the distalmost face of shaved coil 133) abuts a proximal-facing surface of proximal end 139 of clevis 138, such that the distalmost face of coil 130 cannot move distally. In the arrangement shown in FIG. 3A, the position of the distalmost face of coil 130 inhibits any distal movement of coil 130 relative to clevis 138 and other portions of end effector assembly 118. Ring 134 therefore cannot translate within gap 135b. In other embodiments, a space may be between a distalmost face of coil 130 (i.e., the distalmost face of shaved coil 133) and a proximal-facing surface of proximal end 139 of clevis 138, and another space may be between the distal end of covering 131 and shaft connector 132. In these embodiments, ring 134 may translate distally into gap 135b.

A proximal surface of annular shaft connector 132 may abut, or be just distal to, a distal surface of covering 131 and a distally-facing surface of a winding of coil 130. Shaft connector 132 is not fixedly coupled to covering 131 or coil 130. Shaft connector 132 moves distally and proximally (translates axially) relative to shaft 116 within gap 135a. A distal surface of shaft connector 132 is fixedly coupled to a proximal end of clevis 138 by an adhesive, a press-fit, ultrasonic welding, laser welding, or any other means commonly known in the art so that shaft connector 132 moves (including rotation) with clevis 138.

Shaft connector 132 is generally cylindrical and may be protruding radially inward from an inner surface of the end effector assembly. Shaft connector 132 may include a ledge 140 extending radially inward and distally toward gap 135a. Shaved coils 133 may be machined such that coil 130 can also interface with a surface of ledge 140 (i.e., a base or at least a portion of ledge 140). However, ledge 140 is not fixed to shaved coils 133 (i.e., unlike ring 134). For example, ledge 140 of shaft connector 132 may be introduced into gap 135a, and shaft connector 132 may abut the outer surface of shaved coils 133. Furthermore, a surface of shaft connector 132 may abut and be coupled (e.g., welded) to a shoulder 144 of proximal end 139 of clevis 138. In an embodiment, shaft connector 132 and clevis 138 may be manufactured integrally as one part.

Cable 126, and particularly its wire 127, may be fixedly coupled to a distal actuator 136 by various means, including adhesives, a press-fit, ultrasonic welding, laser welding, or any other means commonly known in the art. Distal actuator 136 extends past the distalmost end of coil 130 and proximal end 139 of clevis 138. For example, distal actuator 136 extends such that a distal end of distal actuator 136 is coupled to one or more link(s) 156 (in this embodiment, two links 156) by using a pin 154, permitting pivoting of links 156 relative to distal actuator 136. Further, links 156 may be coupled to a pair of jaws 160 by using one or more axle(s) 158, permitting pivoting of each jaw 160 relative to its associated link 156. A pin 162 is fixedly coupled to clevis 138 and moveably coupled to jaws 160 such that jaws 160 rotate about pin 162.

The combination, of and connections between, the aforementioned components is such that axial translation of cable 126 (and its wire 127) toward distal end 120 may actuate (e.g., open) jaws 160. For example, axial translation of cable 126 may translate actuator 136 such that one of links 156 moves in one direction relative to its axis (e.g., upward) and the other of links 156 moves in the other direction relative to its axis (e.g., downward) such that jaws 160 open about axle 158. Alternatively, axial translation of cable 126 away from distal end 120 may de-actuate (e.g., close) jaws 160.

As described above, distal end effector assembly 118 and cable 126 may be rotated by actuator 112, which is coupled to a proximal portion of wire 127. As described above, a distal end of cable 126 is fixedly coupled to a proximal end of distal actuator 136. The combination and connections between the aforementioned components are such that when cable 126 is rotated by actuator 112, distal actuator 136, clevis 138, links 156, jaws 160, and shaft connector 132 rotate relative to shaft 116. Ring 134, and its relatively movable relationships with shaft connector 132 and clevis 138, permits the relative rotation and maintains the components of end effector assembly 118 (e.g., clevis 138) coupled to components of shaft 116. In other words, ring 134 helps distal end effector assembly 118 remain coupled to shaft 116, regardless of rotational or axial forces. For example, if a force causes shaft 116 to be pulled proximally, ring 134 will move within gap 135a until ring 134 abuts the distal-facing surface of ledge 140 of shaft connector 132, acting as a stop from further proximal movement. In other words, ring 134 acts as a stop and engages a distally-facing surface of shaft connector 132.

Aspects of the disclosure include methods of using device 100. To do so, the user may first introduce the distal end 120 of device 100 into a GI tract via a natural orifice. The orifice can be, for example, the nose, mouth, or anus, and the placement can be in any portion of the GI tract, including the esophagus, stomach, duodenum, large intestine, or small intestine. Delivery and placement also can be in other body lumens or organs reachable via the GI tract, any other natural opening or body tract, bodily incision, or through a delivery device, such as an endoscope or sheath. Once the desired site is accessed, the user can actuate one or more actuators, including actuators 108 and 112, to control the actuation and/or the rotation of the distal end effector assembly 118 relative to the shaft 116 of the medical device 100.

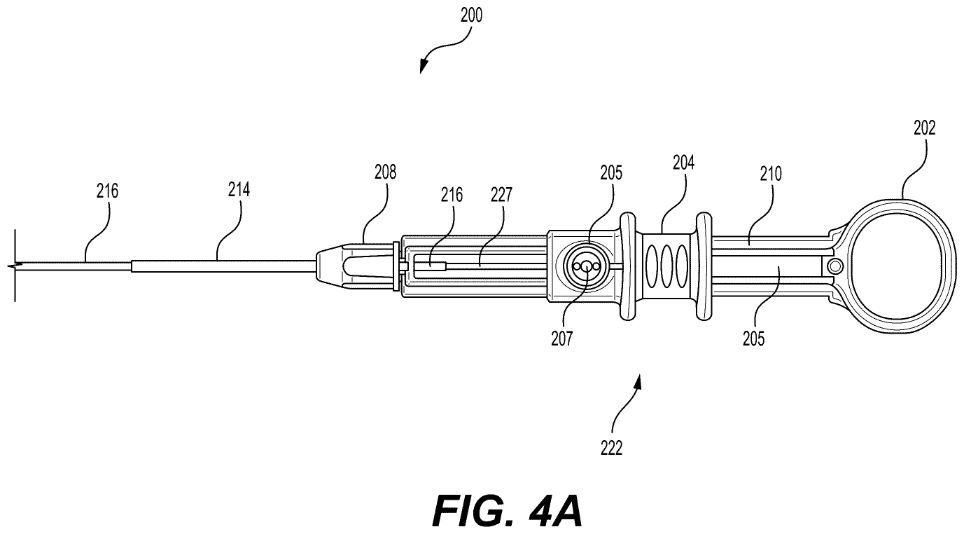

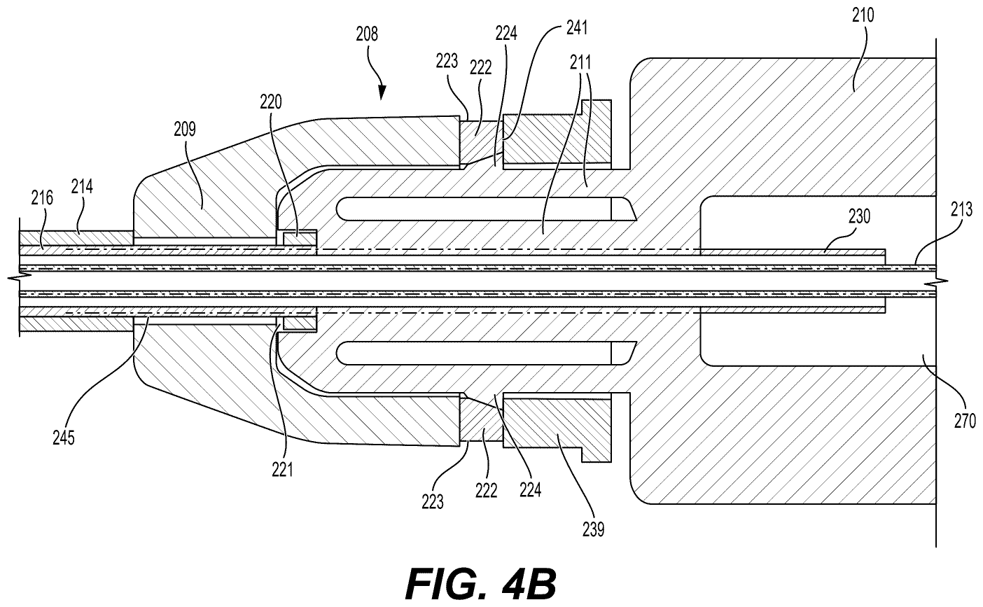

FIG. 4A is a general depiction of a medical device 200 in accordance with examples of this disclosure. As illustrated in FIG. 4A, medical device 200 is similar to medical device 100, except as described herein. More particularly, the shaft and distal components of medical device 200 may be the same as those for medical device 100. The handle components of the two devices may be the same, except as described further herein. Moreover, the embodiment of FIGS. 4A-4B may also use the exemplary method described above, except as shown and described below.

Similar to medical device 100, medical device 200 comprises a proximal component including a handle assembly 233, which includes a handle piece 210 and a finger slot 202 located at the proximal-most end of handle piece 210, and a distal component including a shaft 216 and a conductive wire 227. However, medical device 200 does not include actuator 112. Medical device 200 includes different mechanisms and features to control the rotation and actuation of, for example, conductive wire 227 and the distal end effector assembly.

Handle piece 210 includes an actuator 204 in the form of a spool. Actuator 204 may be coupled to handle piece 210. Actuator 204 may include an indented portion with a relatively smaller lateral cross-section, and extended portions with a relatively larger lateral cross-section. The indented portion may receive a user's fingers, allowing the user to control the movement of actuator 204. Handle piece 210 may include a slot 270 that extends longitudinally through a portion of handle piece 210, starting from a position spaced distally from finger slot 202. Actuator 204 is movable along slot 270, allowing for longitudinal movement either distally or proximally. For example, the movement of actuator 204 may result in the actuation or de-actuation of distal end effector assembly 118 via conductive wire 227 (i.e., similar to conductive wire 127), as described above.

Actuator 204 may include an electrical insert 207. Electrical insert 207 may be within a protrusion 205 that extends radially from a surface of actuator 204. Electrical insert 207 and protrusion 205 may be located at or near the distal end of actuator 204. The proximal portion of the conductive wire 227 may extend into actuator 204 and be fixedly coupled to electrical insert 207 by means of a crimp, glue, or other means in the art. Conductive wire 227 may then extend distally to a distal end of medical device 200, as described below.

The configuration and connections between conductive wire 227, electrical insert 207, actuator 204, and handle piece 210 help enable the rotation of conductive wire 227 when handle piece 210 rotates. For example, when a user rotates handle piece 210 clockwise, conductive wire 227, electrical insert 207, and actuator 204 rotate accordingly. In other words, rotation of handle piece 210 causes conductive wire 227 to rotate because conductive wire 227 is fixedly coupled to electrical insert 207 (i.e., also coupled to actuator 204), which is coupled to handle piece 210. As discussed in further detail below, the above components may rotate relative to a holder 208 and a shaft 216.

FIG. 4B is a cross-sectional view of the distal end of handle piece 210 and an exemplary holder 208. The components of holder 208 and the distal components of handle piece 210 facilitate the rotation of conductive wire 227 and handle piece 210 about its axis, relative to holder 208 and shaft 216 of the medical device 200, as described below.

Handle piece 210 may include an extension 211 that extends distally from a midpoint of handle piece 210. Extension 211 may taper at its distal end to a narrow point and have a rounded shape at its most distal end. Additionally, a portion of extension 211 may include protrusion(s) 224, for example, on both sides of extension 211. Protrusion(s) 224 may extend radially outward relative to an outer surface of extension 211, to engage a portion of holder 208, as discussed below. Handle piece 210 may also define an annular gap 221 at a distal end of extension 211, sized to accommodate an annular ring 220 within gap 221. Annular ring 220, or any similarly shaped circular cross section, attaches to shaft 216, matches the shape of gap 221 to sit in gap 221 without being fixed to extension 211. This will prevent rotation of shaft 216, when handle piece 210 is rotated. Annular ring 220 may have an inner diameter that matches or approximately matches the outer diameter of shaft 216.

Holder 208 may be a cylindrical structure with a cross-sectional width decreasing distally. Holder 208 may include a proximal portion 239 and a nose portion 209. Nose portion 209 may taper to a narrow point at its most distal end. Holder 208 also includes a cavity 249 having a size and shape to receive extension 211, such that extension 211 may be nested within cavity 249. Nose portion 209 is coupled to proximal portion 239 of holder 208. For example, a connector portion 223 may connect nose portion 239 and a distal surface 241 of proximal portion 239. In an embodiment, nose portion 239, connector portion 223, and proximal portion 239 are a one-piece integral structure. Connector portion 223 may be shaped or positioned to define one or more opening(s) 222 between proximal portion 239 and nose portion 209 of holder 208. Openings may extend circumferentially about extension 211, between connector portions 223.

Holder 208 may include a shaft opening 245 extending longitudinally through nose portion 209, as shown in FIG. 2B. Shaft opening 245 receives shaft 206 and wire 227 therein). A strain relief 214 that may extend into or abut a surface of nose portion 209. Additionally, the distal end of handle piece 210, particularly extension 211, includes a wire channel 215. Wire channel 215 extends distally from slot 270 to the distalmost end of handle piece 210 (i.e., a distalmost end of extension 211) and longitudinally at a center of extension 211, to receive wire 227.

As shown in FIG. 4B, protrusion(s) 224 of extension 211 may extend radially outward into opening(s) 222 of holder 208. Once within opening(s) 222, protrusion(s) 224 may abut or engage a distal surface 241 of proximal portion 239, to inhibit removal of handle piece 210 from holder 208. Protrusion(s) 224 are not fixedly coupled to holder 208 (i.e., proximal portion 239), but may longitudinally hold handle piece 210 relative to holder 208. Protrusion(s) 224 inhibit holder 208 from moving distally relative to handle piece 210. As further discussed below, because protrusion(s) 224 are not fixedly attached to holder 208, protrusion(s) may rotate within openings 222. This design may enable extension 211, and consequently handle piece 210, to rotate relative to holder 208.

The position of shaft 216 and conductive wire 227 within extension 211 of handle piece 210 and holder 208 will now be described. Shaft 216 may extend proximally through strain relief 114 and shaft opening 245 of holder 208. The proximal-most end of shaft 216 may extend into and sit within annular gap 221 of extension 211. In the embodiment, annular ring 220 may be coupled to a radially outward-facing surface of shaft 216, helping shaft 216 remain secured within annular gap 221. The radial width of annular gap 221 may be equal to or slightly less than the radius of annular ring 220, forming a stable seating area that helps prevent longitudinal movement of ring 220 and shaft 216 relative to holder 208 and handle piece 210. For example, when a distal force is applied to handle piece 210, annular ring 220 can abut a surface of nose portion 209, stopping further distal movement of ring 220. Conversely, ring 220 abuts a surface of extension 211 when a proximal force is applied (for example to shaft 216), preventing further proximal movement of ring 220 relative to holder 208 and handle piece 210.

Conductive wire 227 may be radially surrounded by a hypotube 213. Conductive wire 227 may be introduced into extension 211 via a tube 230, which surrounds hypotube 213. Tube 230 may surround hypotube 213 until it abuts an inner surface of handle piece 210, just proximal to the proximal opening of wire channel 215. Once conductive wire 227 extends past holder 208 and extension 211, it continues to the distal end of medical device 200, similar to conductive wire 127 in the previous embodiment.

As described above, a user may rotate a handle piece 210 and conductive wire 227 relative to holder 208 and shaft 216. As described above, conductive wire 227 is fixedly coupled to electrical insert 207 within actuator 204, which is connected to handle piece 210. The combination and connections of these components allow for the rotation of handle piece 210, actuator 204, and conductive wire 227, relative to holder 208 and shaft 216. When the user manually rotates handle piece 210, this rotational force is transferred to extension 211 and its protrusion(s) 224. These protrusions 224 may engage with holder 208 to prevent axial movement but are not fixedly attached, allowing them to rotate within openings 222. This configuration enables handle piece 210 to rotate relative to holder 208. Meanwhile, annular ring 220, positioned on shaft 216, seats shaft 216 within annular gap 221, inhibiting movement of shaft 216 while allowing handle piece 210 (i.e., with conductive wire 227 within its wire channel 215) to rotate.

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

Accordingly, various aspects discussed herein may help to improve the efficacy of treatment, for example, a procedure to treat a treatment site. Various aspects discussed herein may help to reduce and/or minimize the duration of the procedure, may reduce the risks of inadvertent manipulation by the user, and/or may help reduce risks of inadvertent contact with tissue or other material during delivery, repositioning, or usage of a medical device in the procedure.

While principles of this disclosure are described herein with reference to illustrative aspects for various applications, it should be understood that the disclosure is not limited thereto. Those having ordinary skill in the art and access to the teachings provided herein will recognize additional modifications, applications, aspects, and substitution of equivalents all fall within the scope of the aspects described herein. Accordingly, the disclosure is not to be considered as limited by the foregoing description.

What is claimed is:

1. A medical device, comprising:
a shaft;
an end effector assembly at a distal end of the shaft; and
a connection between the end effector assembly and the shaft, permitting axial rotation of the end effector assembly relative to the shaft, the connection including:
a shaft connector fixed to a proximal end of the end effector assembly and protruding radially inward from an inner surface of the end effector assembly; and
a stop fixed to a radially outward facing surface of the shaft, distal of the shaft connector, the stop being axially translatable relative to the shaft connector to engage a distally-facing surface of the shaft connector.

2. The medical device of claim 1, wherein the end effector assembly includes a clevis, wherein a proximal end of the clevis includes a shoulder, and wherein the shaft connector is coupled to the shoulder of the clevis.

3. The medical device of claim 2, wherein a first gap is defined between the stop and the clevis, and wherein a second gap is defined between the stop and the shaft connector.

4. The medical device of claim 3, wherein the shaft connector includes a ledge, wherein the ledge extends radially inward and distally toward the second gap.

5. The medical device of claim 3, wherein the shaft connector translates axially relative to the shaft within the second gap.

6. The medical device of claim 4, wherein the stop rotates relative to the shaft connector.

7. The medical device of claim 4, wherein the stop abuts a distal-facing surface of the ledge when the stop translates axially in a proximal direction.

8. The medical device of claim 2, wherein the end effector assembly rotates when moved by an actuator, and wherein the shaft connector and the clevis rotate relative to the shaft during rotation.

9. The medical device of claim 1, wherein the shaft comprises a coil and a covering, wherein the covering is fixed to an outer surface of a proximal portion of the coil, and wherein a proximal surface of the shaft connector abuts the covering.

10. The medical device of claim 9, wherein a distal portion of the coil has an outer diameter that is less than an outer diameter of the proximal portion of the coil.

11. The medical device of claim 10, wherein the stop has an inner diameter matching the outer diameter of the distal portion of the shaft, wherein the stop is fixed to the distal portion of the coil, and wherein the shaft connector is radially outward of the distal portion of the coil.

12. The medical device of claim 1, wherein the medical device includes a handle piece and an actuator coupled to the handle piece,
wherein a wire is fixedly coupled to the actuator and extends to the end effector assembly for actuation of the end effector assembly, and
wherein the handle piece includes a distal extension having a radial protrusion and an annular gap.

13. The medical device of claim 12, wherein the medical device includes a holder defining a circumferential opening that receives the radial protrusion.

14. The medical device of claim 13, wherein an annular ring is positioned on the shaft and sits within the annular gap in the extension.

15. The medical device of claim 14, wherein the handle piece, the actuator, and the wire rotate relative to the holder and the shaft.

16. A medical device, comprising:
a shaft;
a handle piece coupled to a proximal portion of the shaft;
an end effector assembly at a distal end of the shaft;
a holder surrounding a distal portion of the handle piece and a proximal portion of the shaft; and
a connection between the handle piece, the holder, and the shaft, permitting axial rotation of the end effector assembly relative to the shaft, the connection including:
a protrusion extending radially outward from the handle piece;
an opening in the holder, wherein the protrusion extends radially outward into the opening; and
an annular ring fixed to a radially outward facing surface of the shaft, wherein the ring seats the shaft within the handle piece.

17. The medical device of claim 16, further comprising an actuator coupled to the handle piece, wherein the handle piece includes a distal extension having the protrusion and defining an annular gap at a distal end of the extension.

18. The medical device of claim 17, wherein a wire is fixedly coupled to the actuator and extends through the shaft to the end effector assembly for actuation of the end effector assembly.

19. The medical device of claim 17, wherein the holder comprises a proximal portion and a nose portion, wherein the nose portion tapers distally and defines a cavity configured to receive the distal extension of the handle piece.

20. The medical device of claim 19, wherein the annular ring abuts a surface of the nose portion and inhibits axial movement of the shaft relative to the handle piece.
